(19) United States
(12) Patent Application Publication
(10) Pub. No.: US 2025/0376429 A1
(43) Pub. Date: Dec. 11, 2025

(54) METHOD FOR PREPARING 2,5-HEXANEDIONE USING 5-CHLOROMETHYLFURFURAL

(71) Applicant: Xiamen University, Xiamen (CN)

(72) Inventors: Xianhai ZENG, Xiamen (CN); Binglin CHEN, Xiamen (CN); Renjie HUANG, Xiamen (CN); Zhendong YU, Xiamen (CN); Zheng LI, Xiamen (CN); Xing TANG, Xiamen (CN); Lu LIN, Xiamen (CN)

(21) Appl. No.: 19/288,676

(22) Filed: Aug. 1, 2025

Related U.S. Application Data
(63) Continuation of application No. PCT/CN2024/075110, filed on Feb. 1, 2024.

(30) Foreign Application Priority Data
Feb. 10, 2023 (CN) 202310101729.7

(51) Int. Cl. C07C 45/59

(52) U.S. Cl. CPC C07C 45/59

(57) ABSTRACT

A method for preparing 2,5-hexanedione using 5-chloromethylfurfural, the method includes the following steps: adding the 5-chloromethylfurfural, a catalyst, polymethylhydrosiloxane, tetrahydrofuran, and water into a reaction vessel to catalyze a synthesis of the 2,5-hexanedione from the 5-chloromethylfurfural.

### RELATED APPLICATIONS

This application is a continuation of International patent application PCT/CN2024/075110, filed on Feb. 1, 2024, which claims priority to Chinese patent application 202310101729.7, filed on Feb. 10, 2023. International patent application PCT/CN2024/075110 and Chinese patent application 202310101729.7 are incorporated herein by reference.

### FIELD OF THE DISCLOSURE

The present disclosure relates to the field of organic synthesis, and specifically relates to a method for preparing 2,5-hexanedione using 5-chloromethylfurfural.

### BACKGROUND OF THE DISCLOSURE

2,5-hexanedione (HD) is a promising biomass-derived platform chemical with great industrial applications. HD is used as a high-boiling solvent in synthetic resins, nitro lacquers, colorants, printing inks, etc., and in numerous fields such as leather tanning agents, rubber vulcanization accelerators, and manufacture of pesticides and pharmaceuticals (ACS Catal., 2020, 10, 4261-4267).

Currently, the preparation of HD by hydrogenation and hydrolysis of biomass-based platform molecule 5-hydroxymethylfurfural (HMF) has problems such as low yield (8-50%) and long reaction time (1-24 hours) (ChemSusChem, 2022, 15, e202102444; J. Catal., 2019, 375, 224-233; Green Chem., 2016, 18 (10), 3075-3081). In addition, HMF is mainly prepared from fructose, which has a high cost, and active chemical properties and hydrophilicity of HMF make subsequent separation and purification difficult, further limiting large-scale preparation of HD using HMF as the raw materials. Paths for synthesizing HD based on downstream products of HMF have been reported, such as preparing HD by hydrolysis and hydrogenation of 5-methylfurfural/5-methylfurfuryl alcohol (ACS Catal., 2020, 10 (7), 4261-4267), preparing HD by hydrolysis of 2,5-dimethylfuran (ChemSusChem, 2014, 7 (8), 2089-2093), and preparing HD by oxidation of 2,5-hexanediol (Synlett, 2014, 25 (19): 2757-2760). Although HD (with a yield of 70-90%) can be efficiently synthesized using the downstream products of HMF as the raw materials, there are problems such as high costs and complex processes in raw material preparation. Therefore, seeking a low-cost, efficient, and simple process for preparing HD is a prerequisite for mass production and industrialization of HD.

5-chloromethylfurfural (CMF), as a new biomass-based platform molecule, can be directly prepared in high yield under mild conditions from raw materials, such as cellulose and biomass, and characteristics such as stability and hydrophobicity of CMF facilitate subsequent separation and purification of CMF (ACS Sustain. Chem. Eng., 2019, 7 (6), 5588-601). However, there are rare reports on a synthesis of HD from CMF.

### BRIEF SUMMARY OF THE DISCLOSURE

An objective of the present disclosure is to provide a method for preparing 2,5-hexanedione (HD) using raw materials such as polymethylhydrosiloxane and 5-chloromethylfurfural (CMF) with short reaction time and high yield to address problems of high cost and harsh reaction conditions in the existing preparation of the 2,5-hexanedione.

The technical solution of the present disclosure is as follows. Polymethylhydrosiloxane (PMHS) is used hydrogen source, 2,5-hexanedione is prepared from the 5-chloromethylfurfural by catalyzing, a reaction mechanism is as follows.

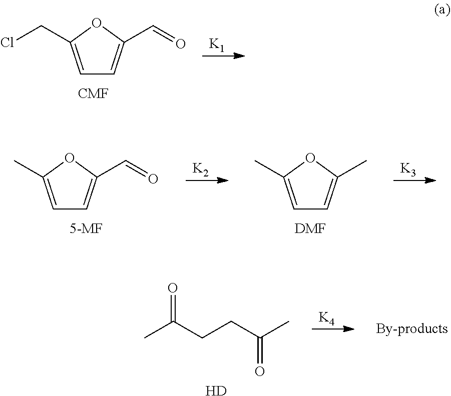

Specifically, the method comprises the following steps:
adding the 5-chloromethylfurfural, a catalyst, polymethylhydrosiloxane, tetrahydrofuran, and water into a reaction vessel to catalyze a synthesis of the 2,5-hexanedione from the 5-chloromethylfurfural.

In a specific embodiment, the reaction vessel is a closed vessel. The reaction vessel preferably a thick-walled pressure-resistant bottle.

In a specific embodiment, the catalyzing the synthesis of the 2,5-hexanedione from the 5-chloromethylfurfural comprises catalyzing the synthesis of the 2,5-hexanedione from the 5-chloromethylfurfural at a temperature of 100-200° C. with a stirring speed of 400-1000 revolutions per minute (rpm) for 5-300 minutes. Preferably, the catalyzing the synthesis of the 2,5-hexanedione from the 5-chloromethylfurfural comprises catalyzing the synthesis of the 2,5-hexanedione from the 5-chloromethylfurfural at a temperature of 140-160° C. with a stirring speed of 500-700 revolutions per minute (rpm) for 30-60 minutes. More preferably, the catalyzing the synthesis of the 2,5-hexanedione from the 5-chloromethylfurfural comprises catalyzing the synthesis of the 2,5-hexanedione from the 5-chloromethylfurfural at a temperature of 160° C. with a stirring speed of 600 revolutions per minute (rpm) for 30 minutes.

In a specific embodiment, a ratio of the 5-chloromethylfurfural, the polymethylhydrosiloxane, the tetrahydrofuran, and the water is 0.072 g: 0.05-0.3 g: 0.5-5 mL: 0.1-3 mL.

In a specific embodiment, a ratio of the 5-chloromethylfurfural, the polymethylhydrosiloxane, the tetrahydrofuran, and the water is 0.072 g: 0.1-0.3 g: 1-2.5 mL: 0.1-1.5 mL.

In a specific embodiment, a ratio of the 5-chloromethylfurfural, the polymethylhydrosiloxane, the tetrahydrofuran, and the water is 0.072 g: 0.2 g: 2.1 mL: 0.4 mL.

In a specific embodiment, the catalyst is Pd/Al₂O₃.

In a specific embodiment, a ratio of the 5-chloromethylfurfural, the Pd/Al₂O₃, the polymethylhydrosiloxane, the tetrahydrofuran, and the water is 0.072 g: 0.005-0.07 g: 0.05-0.3 g: 0.5-5 mL: 0.1-3 mL. Preferably, the ratio of the 5-chloromethylfurfural, the Pd/Al₂O₃, the polymethylhydrosiloxane, the tetrahydrofuran, and the water is 0.072 g: 0.005-0.03 g: 0.01-0.3 g: 1-2.5 mL: 0.1-1.5 mL. More preferably, the ratio of the 5-chloromethylfurfural, the Pd/Al₂O₃, the polymethylhydrosiloxane, the tetrahydrofuran, and the water is 0.072 g: 0.1 g: 0.2 g: 2.1 mL: 0.4 mL.

The present disclosure has the following advantages.

Polymethylhydrosiloxane, as a by-product of the silicon industry, is a low-cost, non-toxic, and stable silicone oil and usually used as a water repellent for various materials such as fabrics, glass, ceramics, paper, leather, metals, cement, and marble, especially for water repellency of the fabrics. In the present disclosure, PMHS is used as a raw material to react with 5-chloromethylfurfural and tetrahydrofuran to prepare 2,5-hexanedione. Thus, a method for directly and efficiently synthesizing HD based on a new biomass-based platform molecule is developed. The present disclosure uses polymethylhydrosiloxane as one of the raw materials to realize an efficient synthesis of 2,5-hexanedione using 5-chloromethylfurfural. 5-chloromethylfurfural can be directly prepared from biomass in high yield with high product selectivity and short reaction time, achieving 100% conversion rate in about 0.5 hours. Therefore, the present disclosure provides a sustainable development path for preparing 2,5-hexanedione using renewable resources.

The method is safe and environmentally friendly. The yield of 2,5-hexanedione in the present disclosure exceeds that of all reported reaction systems using 5-hydroxymethylfurfural as one of the raw materials, thus possessing great industrial application value.

### BRIEF DESCRIPTION OF THE DRAWING

FIG. 1 illustrates a gas chromatography-mass spectrometry (GC-MS) spectrum of 2,5-hexanedione prepared in Embodiment 1 of the present disclosure.

### DETAILED DESCRIPTION OF THE EMBODIMENTS

The present disclosure will be further described below in combination with the embodiments. Unless otherwise specified, the reagents and instruments used in the following embodiments are commercially available products. The specific embodiments are as follows:

### Embodiment 1

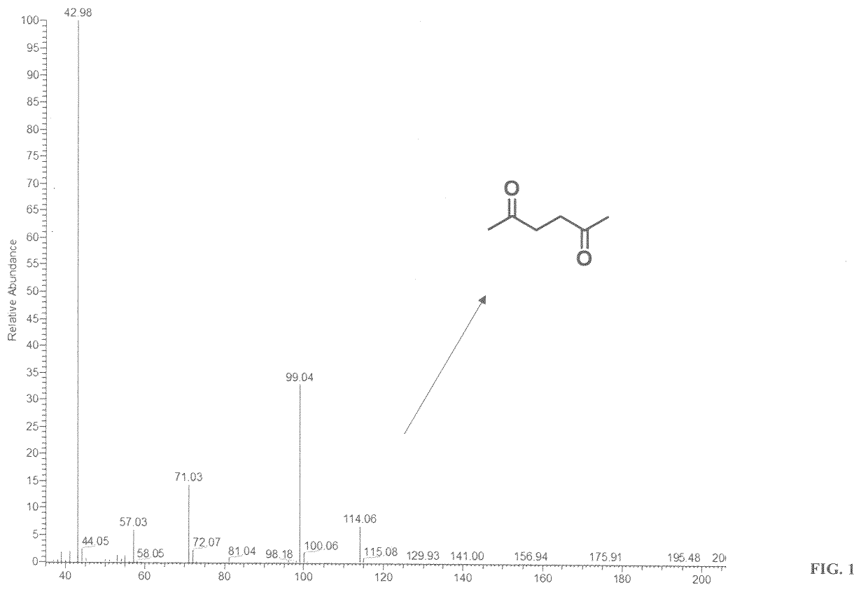

0.072 g of 5-chloromethylfurfural (CMF), 0.010 g of Pd/Al₂O₃ as a catalyst, 0.2 g of polymethylhydrosiloxane (PMHS), 2.1 mL of tetrahydrofuran (THF), and 0.4 mL of water are added into a thick-walled pressure-resistant bottle and reacted at 160° C. with a stirring speed of 600 revolutions per minute (rpm) for 30 minutes. After the reaction, solid-liquid separation is performed using a centrifuge (8000 revolutions/minute (r/min) for 5 minutes), and quantitative analysis is performed using a gas chromatograph (GC, Agilent 7890B). Qualitative analysis is performed using gas chromatography-mass spectrometry (GC-MS, Thermo Scientific). FIG. 1 is the gas chromatography-mass spectrometry spectrum of 2,5-hexanedione (HD) prepared in Embodiment 1 of the present disclosure. The result is as follows: the molar yield of the 2,5-hexanedione is 78%.

### Embodiment 2

The reaction is performed according to the method of Embodiment 1 and differs from Embodiment 1 in that 2.0 mL of tetrahydrofuran (THF) and 0.5 mL of water are used, and the result is as follows: the molar yield of 2,5-hexanedione is 76%.

### Embodiment 3

The reaction is performed according to the method of Embodiment 1 and differs from Embodiment 1 in that 0.3 g of polymethylhydrosiloxane (PMHS) is used, and the result is as follows: the molar yield of the 2,5-hexanedione is 74%.

### Embodiment 4

The reaction is performed according to the method of Embodiment 1 and differs from Embodiment 1 in that 0.03 g of the Pd/Al₂O₃ is used, and the result is as follows: the molar yield of the 2,5-hexanedione is 75%.

### Embodiment 5

The reaction is performed according to the method of Embodiment 1 and differs from Embodiment 1 in that 0.036 g of 5-chloromethylfurfural (CMF) is used, and the result is as follows: the molar yield of the 2,5-hexanedione is 74%.

### Embodiment 6

The reaction is performed according to the method of Embodiment 1 and differs from Embodiment 1 in that the reaction is performed at 140° C. for 90 minutes, and the result is as follows: the molar yield of the 2,5-hexanedione is 76%.

The results described above are summarized in the following table:

TABLE 1
Effects of different types of catalysts and process variables on the
yield of 5-chloromethylfurfural using hydrogenation and hydrolysis

| | | PMHS | Pd/Al₂O₃ | CMF | Temperature | Time | HD Yield |
|---|---|---|---|---|---|---|---|
| Embodiment | V_{THF}:V_{H}_{₂}_{O} | (g) | (g) | (g) | (° C.) | (minute) | (%) |
| 1 | 2.1:0.4 | 0.2 | 0.01 | 0.072 | 160 | 30 | 78 |
| 2 | 2.0:0.5 | 0.2 | 0.01 | 0.072 | 160 | 30 | 76 |
| 3 | 2.1:0.4 | 0.3 | 0.01 | 0.072 | 160 | 30 | 74 |
| 4 | 2.1:0.4 | 0.2 | 0.03 | 0.072 | 160 | 30 | 75 |
| 5 | 2.1:0.4 | 0.2 | 0.01 | 0.036 | 160 | 30 | 74 |
| 6 | 2.1:0.4 | 0.2 | 0.01 | 0.072 | 140 | 90 | 76 |

### COMPARATIVE EXPERIMENTS AND RESULTS

To further illustrate the effects of the present disclosure, Table 2 summarizes an effect comparison between the method of the present disclosure and the existing technology. Table 2 illustrates a comparison between the present disclosure and the existing literature using HMF as a raw material. It can be clearly seen that the HD yield of the present disclosure using CMF is significantly higher than that using HMF as the raw material, the method of the present disclosure using CMF as the raw material has more industrial application prospects, and the present disclosure has great industrial application prospects.

TABLE 2
Comparison between the present disclosure and the literature using HMF as the raw material

| | | Temper- | | | | Conver- | | |
|---|---|---|---|---|---|---|---|---|
| | | ature | Time | Hydrogen | Raw | sion | Yield | |
| No. | Catalyst | (° C.) | (hour) | Donor | Material | (%) | (%) | Reference |
| 1 | PdI/Al₂O₃ | 170 | 6 | 6 Mpa H₂ | HMF | 98 | 50 | ChemSusChem, |
| | | | | | | | | 2022, 15, |
| | | | | | | | | e202102444 |
| 2 | Pd/C + | 70 | 1 | 4 Mpa H₂ | HMF | 92 | 11 | Molecules, 2020, |
| | Acetic Acid | | | | | | | 25(11): 2475. |
| 3 | Pd/Beta | 110 | 24 | 6 Mpa H₂ | HMF | 100 | 8 | J. Catal., 2019, |
| | | | | | | | | 375, 224-233 |
| 4 | Ni/CN + | 200 | 3 | 2 Mpa H₂ | HMF | 99.4 | 26 | ACS Sustain. |
| | Phosphoric | | | | | | | Chem. Eng., |
| | Acid | | | | | | | 2021, 9(46), |
| | | | | | | | | 15394-15405 |
| 5 | Zn | 250 | 2.3 | Zn/H₂O | HMF | 100 | 27 | Green Chem., |
| | | | | | | | | 2016, 18(10), |
| | | | | | | | | 3075-3081 |
| 6 | Pd/Al₂O₃ | 160 | 0.5 | PMHS | CMF | 100 | 78 | Embodiment 1 |

The embodiments are merely used for an objective of providing exemplary descriptions, and the protective scope of the present disclosure is not limited thereto in any way. Thus, it is intended that the protective scope of the present disclosure cover improvements and modifications provided they are improved or modified based on the aforementioned descriptions by a person skilled in the art.

What is claimed is:

1. A method for preparing 2,5-hexanedione using 5-chloromethylfurfural, comprising:
adding the 5-chloromethylfurfural, a catalyst, polymethylhydrosiloxane, tetrahydrofuran, and water into a reaction vessel to catalyze a synthesis of the 2,5-hexanedione from the 5-chloromethylfurfural.

2. The method according to claim 1, wherein the reaction vessel is a closed vessel.

3. The method according to claim 1, wherein:
the catalyzing the synthesis of the 2,5-hexanedione from the 5-chloromethylfurfural comprises catalyzing the synthesis of the 2,5-hexanedione from the 5-chloromethylfurfural at a temperature of 100-200° C. with a stirring speed of 400-1000 revolutions per minute (rpm) for 5-300 minutes.

4. The method according to claim 1, wherein:
the catalyzing the synthesis of the 2,5-hexanedione from the 5-chloromethylfurfural comprises catalyzing the synthesis of the 2,5-hexanedione from the 5-chloromethylfurfural at a temperature of 140-160° C. with a stirring speed of 500-700 revolutions per minute (rpm) for 30-60 minutes.

5. The method according to claim 1, wherein:
the catalyzing the synthesis of the 2,5-hexanedione from the 5-chloromethylfurfural comprises catalyzing the synthesis of the 2,5-hexanedione from the 5-chloromethylfurfural at a temperature of 160° C. with a stirring speed of 600 revolutions per minute (rpm) for 30 minutes.

6. The method according to claim 1, wherein a ratio of the 5-chloromethylfurfural, the polymethylhydrosiloxane, the tetrahydrofuran, and the water is 0.072 g: 0.05-0.3 g: 0.5-5 mL: 0.1-3 mL.

7. The method according to claim 1, wherein a ratio of the 5-chloromethylfurfural, the polymethylhydrosiloxane, the tetrahydrofuran, and the water is 0.072 g: 0.1-0.3 g: 1-2.5 mL: 0.1-1.5 mL.

8. The method according to claim 1, wherein a ratio of the 5-chloromethylfurfural, the polymethylhydrosiloxane, the tetrahydrofuran, and the water is 0.072 g: 0.2 g: 2.1 mL: 0.4 mL.

9. The method according to claim 1, wherein the catalyst is Pd/Al₂O₃.

10. The method according to claim 9, wherein a ratio of the 5-chloromethylfurfural, the Pd/Al₂O₃, the polymethylhydrosiloxane, the tetrahydrofuran, and the water is 0.072 g: 0.005-0.07 g: 0.05-0.3 g: 0.5-5 mL: 0.1-3 mL.

11. A method for preparing 2,5-hexanedione using 5-chloromethylfurfural, comprising:
adding the 5-chloromethylfurfural, a catalyst, polymethylhydrosiloxane, tetrahydrofuran, and water into a reaction vessel to catalyze a synthesis of the 2,5-hexanedione at a temperature of 100-200° C. for 5-300 minutes.

12. The method according to claim 11, wherein a ratio of the 5-chloromethylfurfural, the polymethylhydrosiloxane, the tetrahydrofuran, and the water is 1 g: 0.69-4.16 g: 6.94-69.4 mL: 1.39-41.7 mL.

13. The method according to claim 11, wherein the catalyst is Pd/Al₂O₃.
